(19) United States
(12) Patent Application Publication
(10) Pub. No.: US 2019/0110754 A1
(43) Pub. Date: Apr. 18, 2019

(54) MACHINE LEARNING BASED SYSTEM FOR IDENTIFYING AND MONITORING NEUROLOGICAL DISORDERS

(71) Applicants: Satish Rao, Boulder, CO (US); Matthew Wilder, Boulder, CO (US)

(72) Inventors: Satish Rao, Boulder, CO (US); Matthew Wilder, Boulder, CO (US)

(21) Appl. No.: 16/162,711

(22) Filed: Oct. 17, 2018

Related U.S. Application Data
(60) Provisional application No. 62/573,622, filed on Oct. 17, 2017.

(51) Int. Cl. A61B 5/00; G06N 3/08; G06N 5/02; G06N 7/00

(52) U.S. Cl. CPC A61B 5/7267; A61B 5/0015; A61B 5/4082; G06N 5/022; G06N 7/00; G06N 3/08

(57) ABSTRACT

A system and methods of diagnosing and monitoring neurological disorders in a patient utilizing an artificial intelligence based system. The system may comprise a plurality of sensors, a collection of trained machine learning based diagnostic and monitoring tools, and an output device. The plurality of sensors may collect data relevant to neurological disorders. The trained diagnostic tool will learn to use the sensor data to assign risk assessments for various neurological disorders. The trained monitoring tool will track the development of a disorder over time and may be used to recommend or modify the administration of relevant treatments. The goal of the system is to render an accurate evaluation of the presence and severity of neurological disorders in a patient without requiring input from an expertly trained neurologist.

### CROSS REFERENCE TO RELATED APPLICATIONS

This application claims priority from U.S. Provisional Patent Application No. 62/573,622, filed Oct. 17, 2017, which is incorporated herein by reference.

### BACKGROUND

The total economic burden of neurologic disease is currently estimated to exceed $800 Billion annually in the United States. Early detection and diagnosis of these diseases typically leads to earlier treatment and a decrease in the total cost of care over an individual's lifetime.

Currently, diagnosis of such diseases requires the involvement of a physician. In the United States, it is predicted that there will be a shortage of between 90,000 and 140,000 physicians by the year 2025. Worldwide, the shortfall is expected to exceed 12.9 Million healthcare providers by 2035.

Furthermore, many general practitioner (GP) physicians lack the necessary training to accurately diagnose movement disorders. For instance, a 1999 study conducted in Britain found that GPs had an error rate of just under 50% when diagnosing Parkinson's disease. (Jolyon Meara et. al., Accuracy of Diagnosis in Patients with presumed Parkinson's disease; Age and Ageing (1999); 28:99-102.). This state of affairs is partially due to the fact that with most movement disorders, the symptoms at onset may be very subtle, and there is typically no obvious trauma to the patient (such as a blow to the head) which would lead the GP to suspect a problem with the patient's nervous system.

While neurologists specializing in the disease are much more accurate in their diagnoses, even general neurologists have a significant error rate. As such there is a need for a diagnostic system that can accurately diagnose a neurological disorder, thus reducing the burden on our medical system by both aiding GPs in making an initial diagnosis and reducing the loss and suffering that result from a potential misdiagnosis.

Additionally, many patients suffering from such diseases are located in remote areas, or otherwise find it difficult to access a trained neurologist to secure an accurate diagnosis of their disease. Thus there is a need for some system of rendering an accurate diagnosis that can be used in a simple clinic setting, or even in the patient's own home, by otherwise untrained individuals.

In addition to movement disorders, dizziness is a common and difficult symptom to diagnose. The prevalence of dizziness and related complaints, such as vertigo and unsteadiness maybe between 40%-50% (Front Neurol. 2013;4:29). Dizziness as a chief complaint in the emergency department (ED) is near 3.9 million visits annually and dizziness can be a component symptom of up to 50% of all ED visits. In terms of the primary care office, there are an approximated 8 million visits annually with the chief complaint of dizziness and 50% of the elderly population will seek medical attention for dizziness.

The challenge for the clinician is twofold: one in the broad use of the word “dizzy” by the patient and second because of the wide range of root causes that can manifest those symptoms. The range of root causes from being benign (common cold) to deadly (stroke).

People very commonly use the word for dizzy as a catch-all word for a variety of more specific symptoms, such as vertigo (hallucination of motion), presyncope (light headedness) or ataxia (lack of balance or coordination). Often the patient themselves, even with skilled probing from the doctor, will not be specific and revert to using the word ‘dizzy’.

The other primary challenge related to the wide variety of causes of dizziness. These maybe due to inner ear/vestibular (benign paroxysmal positional vertigo, vestibular neuronitis, Meniere's disease), neurologic (acute stroke, brain tumor), cardiac (heart failure, low blood pressure), psychiatric (anxiety) and variety of other medical disorders.

A secondary challenge, especially for physicians (commonly emergency physicians, neurologists and internal medicine hospitalists) providing acute care in the emergency department, urgent care, clinics, or hospital is the physical exam. This is centered on discriminating normal from abnormal eye movements. Indeed, even seasoned neurologists can have difficulty accurately examining eye movements. There can also be very subtle abnormalities in motor speech production or facial symmetry.

It is the above three challenges that finally coalesce into the acute evaluation: Is this dizziness life threatening or not? A dangerous cause of dizziness that is difficult to diagnose solely on history and physical exam is acute stroke effecting the posterior circulation.

Indeed, there is data showing that strokes effecting the posterior circulation (vertebro-basilar system supplying blood to the brainstem and back of the brain) are more often missed in the ED than strokes occurring in the anterior circulation (carotid system supply blood to the front of the brain). (Stroke. 2016;STROKEAHA.115.010613)

Furthermore, physicians have a difficult time quickly and accurately diagnosing epileptic seizures. An epileptic seizure is a brief electrical event (mean duration ˜1 minute) that occurs in the cerebral cortex and is caused by an excessive volume of neurons depolarizing (firing') hypersynchronously. One in ten people will have seizure at some point in their life, but only around one in 100 (1%) of the population develop epilepsy. Epilepsy is an enduring propensity towards recurrent, unprovoked seizures.

Sometimes patients have episodes that resemble seizures to the observer but they are not epileptic seizures. These ‘nonepileptic events’ must then be further categorized into physiologic (passing out, heart arrhythmia etc) versus psychogenic. Psychogenic events are the most common diagnostic alternative to epileptic seizures in epilepsy centers, and will be described further.

Psychogenic events are a physiologically different condition that resemble epileptic seizures (ES) to the observer (i.e. following to the ground and convulsing, etc). This disorder, unfortunately, has multiple names in the medical literature adding confusion to patients suffering and nonspecialists treating these conditions. These names include: pseudoseizures, nonepileptic seizures, psychogenic seizures, psychogenic nonepileptic seizures, nonepileptic attack disorder, or nonepileptic behavioral spell.

These terms are synonymous. In this discussion, the preferred term will be nonepileptic behavioral spell (NBS).

Nonepileptic behavioral spells are a psychologic condition that typically stem from a severe emotional trauma prior to the onset of the NBS. In some cases, the trauma may have occurred 40-50 years prior to the onset. The emotional trauma, for unclear reasons, manifests into physical symptoms. This process is broadly termed ‘conversion disorders’ referring to the central nervous system converting emotional pain into physical symptoms. These physical symptoms can often manifest as chronic, unexplained abdominal pain or headaches, for example. Sometimes the emotional pain or stress manifest into episodes of convulsing, or what appears to be alteration of consciousness, these events are NBS.

The gold standard for diagnosing NBS is through inpatient video-electroencephalography (V-EEG) monitoring unit (synonymous term with EMU). This is a time, labor and cost intensive procedure. Patients are typically admitted for three to seven days to the hospital as an inpatient.

Time synchronized digital video, scalp EEG, electrocardiogram (ECG) and pulse oximetry are all recorded continuously 24/7 to record a habitual event.

The diagnosis primarily relies on the ‘ictal EEG’ pattern. Ictal or ictus refers to the event. Therefore, this refers to the what is happening in the brain waves during the actual the episode. For most epileptic seizures, there is a distinct change in the EEG, i.e. the seizure manifests as self-limited rhythmic focal or generalized pattern. There is typically some post-seizure slowing of brain wave frequencies afterwards for a few minutes, and then resumption of normal patterns.

In contrast, during NBS, there is no change in the EEG during the event. There are typically normal background rhythms of wakefulness with superimposed movement/muscle artifacts.

The neurologist considers this ‘ictal EEG’ along with the digital video. Neurologists have long recognized that ES and NBS have distinct differences in their physical manifestations. Furthermore, that with proper education, training and exposure to a high volume of examples, a neurologist can become fairly accurate in diagnosing NBS from digital video or direct observation. These neurologists have usually done a 1-2-year fellowship after neurology residency are termed epileptologists. There is a predicted shortage looming of all neurology providers, including epileptologists.

Even with this body knowledge there can be diagnostic uncertainty in the EMU. For example, there is a type of seizure termed ‘simple partial seizure’ (SPS) that involves only a focal region of the cerebral cortex and does not alter consciousness. Only 15% of SPS will have a distinct ictal EEG pattern. In these cases, the patient's history, imaging and other seizure types are critical to diagnosis. Another example are mesial frontal lobe seizures. These are seizures which originate on the surface of the frontal lobe at midline where the neurons are no longer directly underneath the skull. Ironically, seizures from these regions can create bizarre seizure types (swirling movements, behavioral changes that appear intentional, etc) and, due to the biophysics of EEG, typically due not produce clear ictal EEG changes.

The burden of NBS is large. Approximately 25% of patients referred to specialized epilepsy centers for ‘drug-resistant’ epilepsy are found to actually have NBS. There is average delay of 1-7 years in diagnosing NBS. This leads to unnecessary exposure antiseizure medications, side effects and health care utilization.

An additional challenge is monitoring the progression of a neurological disorder over time. The ability to quantitatively measure this progression could have significant impacts in the development and administration of treatments for these diseases. Additionally, the ability to monitor the state of the disease may enable patients to adjust their treatments without requiring a specialist visit.

As such, there is a need for a system which can, either on its own or in conjunction with a physician, accurately diagnose a specific neurological disorder in a patient without the need for the patient or physician to have any prior training in diagnosing such conditions.

### SUMMARY OF THE INVENTION

It is one aspect of the present invention to provide a system that provides accurate and rapid diagnosis of a patient. In certain embodiments, the system is tailored to diagnose patients presenting with symptoms of a stroke, patients suffering from a potential movement disorder, patients who have recently undergone a seizure, and patients suffering from dizziness.

It is another aspect of the present invention to provide a system that provides useful programing recommendations of medical devices implanted in a patient. In certain embodiments, such programming recommendations will improve therapeutic efficacy of the implanted device, or reduce unwanted side effects. In certain embodiments such implanted medical devices include deep brain stimulation devices (DBSs), which may be implanted to improve symptoms associated with Parkinson's Disease or stroke.

In certain embodiments of the present invention, the system will comprise a series of sensors to collect data from the patient that are relevant to the diagnosis. These sensors may include light sensors, such as video or still cameras, audio sensors, such as those found on standard cellular phones, gyroscopes, accelerometers, pressure sensors, and sensors sensitive to other electromagnetic wavelengths, such as infrared.

In certain embodiments, these sensors will be in communication with an artificial intelligence system. Preferably, this system will be a machine learning system that, once trained, will process the inputs from the various sensors and produce a diagnostic prediction for the patient based on the analysis. This system may then produce an output indicating the diagnosis to the patient or a physician. In some embodiments, the output may be a simple “yes”, “no”, “inconclusive” diagnosis for a particular disease. In alternate embodiments, the output may be a list of the most likely diseases, with a probability score assigned to each one. One key advantage of such a system is that, by training the system to reach a diagnosis in an unbiased manner, the system may be able to identify new clinical indicia of disease, or recognize previously unidentified combinations of symptoms that allow it to accurately diagnose a disorder where even an expert clinician would fail to do so.

In embodiments where the progression of the disease is monitored, the system of the present invention may operate by assigning a “severity” score to a patient and comparing that score to one derived by the system at an earlier timepoint. Such information can be beneficial to a patient, as it allows to the patient to, for example, monitor the success of a course of treatment or determine if a more invasive form of treatment may be justified.

In another aspect of the present invention, the diagnostic system of the present invention is housed in a remotely accessible location, and is capable of performing all of the data processing and analysis necessary to render a diagnosis. Thus in certain embodiments, a physician or patient with limited access to resources or in a remote location may submit raw data collected on the sensors available to them, and receive a diagnosis from the system.

Thus, it is one embodiment of the present invention to provide a system for diagnosing a patient, the system comprising: at least one sensor in communication with a processor and a memory; wherein said at least one sensor in communication with a processor and a memory acquires raw patient data from said patient; wherein said raw patient data comprises at least one of a video recording and an audio recording; a data processing module in communication with the processor and the memory; wherein said data processing module converts said raw patient data into processed diagnostic data; a diagnosis module in communication with the data processing module; wherein said diagnosis module is remote from the at least one sensor; wherein said diagnosis module comprises a trained diagnostic system; wherein said trained diagnostic system comprises a plurality of diagnostic models; wherein each of said plurality of diagnostic models comprise a plurality of algorithms trained to assign a classification to at least one aspect of said processed diagnostic data; and wherein said trained diagnostic system integrates the classifications of said plurality of diagnostic models to output a diagnostic prediction for said patient.

It is another embodiment of the present invention to provide such a system, wherein said diagnosis module is housed on a remote server.

It is yet another embodiment of the present invention to provide such a system, wherein said diagnostic prediction further comprises a confidence value.

It is still another embodiment of the present invention to provide such a system, wherein said at least one sensor is housed within a mobile device.

It is yet another embodiment of the present invention to provide such a system, wherein said trained diagnostic system is trained using a machine learning system.

It is still another embodiment of the present invention to provide such a system, wherein said machine learning system comprises at least one of a convolutional neural network (e.g., Krizhevsky, A., Sutskever, I., and Hinton, G. E. (2012). Imagenet classification with deep convolutional neural networks. In Advances in Neural Information Processing Systems (NIPS 2012)), a recurrent neural network (Jain, L. and Medsker, L. (1999). Recurrent Neural Networks: Design and Applications (1st ed.). CRC Press, Inc., Boca Raton, Fla., USA.), a long-term short-term memory network (Hochreiter, S. and Schmidhuber, J. (1997). Long Short-Term Memory. Neural Comput. 9, 8 (November 1997), 1735-1780.), and a random forest regression model (Breiman, L. (2001). Random Forests. Machine Learning. 45 (1): 5-32.).

It is yet another embodiment of the present invention to provide such a system, wherein said raw patient data comprises a video recording.

It is still another embodiment of the present invention to provide such a system, wherein said video recording comprises a recording of a patient preforming repetitive movements.

It is yet another embodiment of the present invention to provide such a system, wherein said repetitive movements comprise at least one of rapid finger tapping, opening and closing the hand, hand rotations, and heel tapping.

It is still another embodiment of the present invention to provide such a system, wherein said raw patient data comprises an audio recording.

It is yet another embodiment of the present invention to provide such a system, wherein said audio recording comprises the patient reading a prompted sentence aloud.

It is an additional embodiment of the present invention to provide a system for diagnosing a neurological disorder in a patient, the system comprising: at least one sensor in communication with a processor and a memory; wherein said at least one sensor in communication with a processor and a memory acquires raw patient data from said patient; wherein said raw patient data comprises at least one of a video recording and an audio recording, a data processing module in communication with the processor and the memory; wherein said data processing module converts said raw patient data into processed diagnostic data, a diagnosis module in communication with the data processing module; wherein said diagnosis module comprises a trained diagnostic system; wherein said trained diagnostic system comprises a plurality of diagnostic models; wherein each of said plurality of diagnostic models comprise a plurality of algorithms trained to assign a classification to at least one aspect of said processed diagnostic data; and wherein said trained diagnostic system integrates said classifications of said plurality of diagnostic models to output a diagnostic prediction for said patient.

It is another embodiment the present invention to provide such a system, wherein the program executing said diagnosis module is executed on a device that is remote from the at least one sensor.

It is yet another embodiment the present invention to provide such a system, wherein said trained diagnostic system is trained to diagnose a movement disorder.

It is still another embodiment the present invention to provide such a system, wherein said movement disorder is Parkinson's Disease.

It is yet another embodiment the present invention to provide such a system, wherein said raw patient data comprises a video recording, wherein said video recording comprises at least one of: a recording of the patient's face while preforming simple expressions; a recording of the patient's blink rate; a recording of the patient's gaze variations; a recording of the patient while seated; a recording of the patient's face while reading a prepared statement; a recording of the patient preforming repetitive tasks; and a recording of the patient while walking.

It is still another embodiment the present invention to provide such a system, wherein said raw patient data comprises an audio recording, wherein said audio recording comprises at least one of: a recording of the patient repeating a prepared statement; a recording of the patient reading a sentence; and a recording of the patient making plosive sounds.

It is yet another embodiment the present invention to provide such a system, wherein said plurality of algorithms are trained using a machine learning system.

It is still another embodiment the present invention to provide such a system, wherein said machine learning system comprises at least one of: a convolutional neural network; a recurrent neural network; a long-term short-term memory network; support vector machines; and a random forest regression model.

It is another embodiment of the present invention to provide a system for calibrating an implanted medical device in a patient, the system comprising: at least one sensor in communication with a processor and a memory; wherein said at least one sensor in communication with a processor and a memory acquires raw patient data from said patient; wherein said raw patient data comprises at least one of a video recording and an audio recording; a data processing module in communication with the processor and the memory; wherein said data processing module converts said raw patient data into processed calibration data; a calibration module in communication with the data processing module; wherein said calibration module comprises a trained calibration system; wherein said trained calibration system comprises a plurality of calibration models; wherein each of said plurality of calibration models comprise a plurality of algorithms trained to assign a classification to at least one aspect of said processed calibration data; and wherein said trained calibration system integrates said classifications of said plurality of calibration models to output a calibration recommendation for said implanted medical device of said patient.

It is another embodiment of the present invention to provide such a system, wherein the program executing said calibration module is executed on a device that is remote from the at least one sensor.

It is yet another embodiment the present invention to provide such a system, wherein said implanted medical device comprises a deep brain stimulation device (DBS).

It is still another embodiment the present invention to provide such a system, wherein said calibration recommendation comprises a change to the programming settings of said DBS comprising at least one of: amplitude, pulse width, rate, polarity, electrode selection, stimulation mode, cycle, power source, and calculated charge density.

It is yet another embodiment the present invention to provide such a system, wherein said raw patient data comprises a video recording, wherein said video recording comprises at least one of: a recording of the patient's face while preforming simple expressions; a recording of the patient's blink rate; a recording of the patient's gaze variations; a recording of the patient while seated; a recording of the patient's face while reading a prepared statement; a recording of the patient preforming repetitive tasks; and a recording of the patient while walking.

It is still another embodiment the present invention to provide such a system, wherein said raw patient data comprises an audio recording, wherein said audio recording comprises at least one of: a recording of the patient repeating a prepared statement; a recording of the patient reading a sentence; and a recording of the patient making plosive sounds.

It is yet another embodiment the present invention to provide such a system, wherein said plurality of algorithms are trained using a machine learning system.

It is still another embodiment the present invention to provide such a system, wherein said machine learning system comprises at least one of: a convolutional neural network; a recurrent neural network; a long-term short-term memory network; support vector machines; and a random forest regression model.

It is another embodiment of the present invention to provide a system for monitoring the progression of a neurological disorder in a patient diagnosed with such a disorder, the system comprising: at least one sensor in communication with a processor and a memory; wherein said at least one sensor in communication with a processor and a memory acquires raw patient data from said patient; wherein said raw patient data comprises at least one of a video recording and an audio recording; a data processing module in communication with the processor and the memory; wherein said data processing module converts said raw patient data into processed diagnostic data; a progression module in communication with the data processing module; wherein said progression module comprises a trained diagnostic system; wherein said trained diagnostic system comprises a plurality of diagnostic models; wherein each of said plurality of diagnostic models comprise a plurality of algorithms trained to assign a classification to at least one aspect of said processed diagnostic data; wherein said trained diagnostic system integrates said classifications of said plurality of diagnostic models to generate a current progression score for said patient; and wherein said progression module compares said current progression score for said patient to a progression score from said patient generated at an earlier timepoint to create a current disease progression state, and output said disease progression state.

These, and other, embodiments of the invention will be better appreciated and understood when considered in conjunction with the following description and the accompanying tables. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### DESCRIPTION OF THE FIGURES

FIG. 1: Block diagram of one embodiment of the training procedure of the artificial intelligence based diagnostic system.

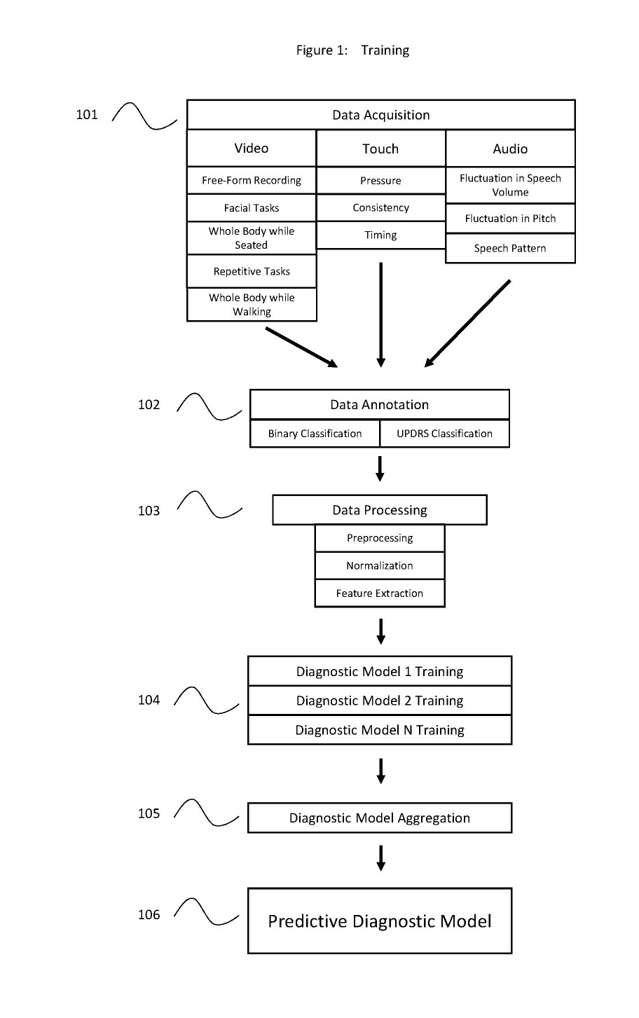

FIG. 2: Block diagram of one embodiment of the diagnostic system as used in practice.

FIG. 3: Diagram illustrating one possible implementation of the system of the present invention.

FIG. 4: Diagram illustrating one possible embodiment of the system of the present invention.

### DETAILED DESCRIPTION OF THE INVENTION

### Definitions

The phrase “comprising at least one of X and Y” refers to situations where X is selected alone, situations where Y is selected alone, and situations where both X and Y are selected together.

A “confidence value” indicates the relative confidence that the diagnostic system has in the accuracy of a particular diagnosis.

A “mobile device” is an electronic device which may be carried and used by a person outside of the home or office. Such devices include, but are not limited to, smartphones, tablets, laptop computers, and PDAs. Such devices typically possess a processor coupled to a memory, an input mechanism, such as a touchscreen or keyboard, and output devices such as a display screen or audio output, and a wired or wireless interface capability, such as wifi, BLUETOOTH™, cellular network, or wired LAN connection that will enable the device to communicate with other computer devices.

A software “module” comprises a program or set of programs executable on a processor and configured to accomplish the designated task. A module may operate autonomously, or may require a user to input certain commands.

A “server” is a computer system, such as one or more computers and/or devices, that provides services to other computer systems over a network.

In certain embodiments, the system consists of a collection of sensors used to record a patient's behaviors over a period of time producing a temporal sequence of data.

The primary system preferably involves utilizing the video and audio sensors commonly available on smart-phones, tablets, and laptops. In addition to these primary sensors, when available, other sensors including range imaging camera, gyroscope, accelerometer, touch screen/pressure sensor, etc. may be used to provide input to the machine learning and diagnostic system. It will be apparent to those having skill in the art that the more sensor data that is available to the system, the more accurate the resulting diagnosis is likely to be once diagnostic systems have been trained using the relevant sensor data.

Thus, in certain embodiments, the purpose of the machine learning system is to take as input the temporal or static data recorded from the sensors and produce as output a probability score for each of a collection of diagnoses. The system may also output a confidence score for each of the diagnostic probabilities. Furthermore, the system may be used to calibrate implanted devices, such as deep brain stimulation devices, to optimize the therapeutic efficacy of such devices.

In light of the challenges described above, one goal of the machine learning system is to serve as an inexpensive means for detecting neurological disorders, including movement disorders. Initially, it is expected that the output of the system will guide physicians in making a decision about a patient, however, this state of affairs may change as confidence grows in the accuracy of the system. As the system will initially be used primarily to identify at-risk patients, it may be tuned to have a low false negative rate (i.e., high sensitivity) at the cost of a higher false positive rate (i.e., lower specificity). In alternate embodiments, the system of present invention may be used to monitor patients after a diagnosis has been made. Such monitoring may be used, for example, to determine disease progression, guide treatment plans for patients, such as recommending dosages of medication to treat a movement disorder, or suggested programing changes for an implanted medical device such as a deep brain stimulation device.

Preferably, the system will include a collection of tests the patient will be asked to perform during which time sensor data will be recorded. These tests will be designed to elicit specific diagnostic information. In certain embodiments, the device used to collect the data will prompt the user or patient to perform the preferable tests. Such prompts may be made, by way of example, by using a written description of the test, by providing a video demonstration to be displayed on the screen of the device (if available), or by providing a frame or other outline on a live video feed displayed on the device to indicate where the camera should be centered. Preferably, the system will be flexible such that it can produce a diagnostic decision without needing results from every test (for example in cases where a particular sensor is unavailable).

In certain embodiments, the patient may repeat the suite of tests at regular or irregular intervals of time. For example, the patient may repeat the test once every two weeks to continually monitor the progression of the disease. In cases where data is collected from multiple points in time, the diagnostic system may integrate across all data points to derive an evaluation of the state of the disease.

In certain embodiments, the machine learning system as a whole will take the data acquired during these tests and use them to produce the desired output. In other embodiments, the system may also integrate background information about a patient including but not limited to age, sex, prior medical history, family history, and results from any additional or alternate medical tests.

The whole machine learning system may include components that utilize specific machine learning algorithms to produce diagnoses from a single test or a subset of the tests. If the system includes multiple diagnostic components, the system will utilize an additional machine learning algorithm to combine across the results in order to produce the final system output. The machine learning system may have a subset of required tests that must be completed for every patient or it can be designed to operate with the data from any available tests. Additionally, the system may prescribe additional tests in order to strengthen the diagnosis.

The processing performed by the machine learning system can be performed on device, on a local desktop machine, or in a remote location via an electronic connection. When processing is not performed on the same device which collected the sensor data, it is assumed that the data will be transmitted to the appropriate computing device, such as a server, using any commonly available wired or wireless technology. It will be apparent to those having skill in the art that in such cases, the remote computer will be configured to receive the data from the initial device, analyze such data, and transmit the result to the appropriate location.

In certain embodiments, the machine learning system for identifying potential diseases comprises one or more machine learning algorithms combined with data processing methods. The machine learning algorithms typically involve several stages of processing to obtain the output including: data preprocessing, data normalization, feature extraction, and classification/regression. The components of the system may be implemented separately for each sensor in which case, the final output results from the fusion of the classification/regression outputs associated with each sensor. Alternatively, some of the sensor data can be fused at the feature extraction stage and passed on to a shared classification/regression model.

In what follows, examples are provided for what each stage of processing entails. This is meant to help elucidate the role of each component, but by no means covers the full range of methods that may be included.

Data preprocessing: Temporally aligning data, subsampling or supersampling (interpolation) in time and space, basic filtering.

Data Normalization: General organization of the data to identify the most important components and to normalize the data across collections. Face detection/localization (e.g., Viola, P. and Jones, M. (2001). Robust real-time face detection. International Journal of Computer Vision (UCV),57(2):137-154.), facial keypoint detection (e.g., Ren, S., Cao, X., Wei, Y., Sun, J. (2014). Face alignment at 3000 fps via regressing local binary features. IEEE Conference on Computer Vision and Pattern Recognition (CVPR), pp. 1685-1692.), speech detection, motion detection.

Feature Extraction: Application of filters or other methods to obtain an abstract feature set that captures the relevant aspects of the input data. An example of this is the extraction of optical flow features from image sequences. In audio, Mel Frequency Cepstral Coefficients (MFCC) might be extracted from the acoustic signal. The feature extraction may be implicitly implemented within the classification/regression model (this is commonly the case with deep learning methods). Alternately, feature extraction may performed prior to passing the data to an artificial neural network.

Classification/Regression: A supervised machine learning algorithm that is trained from data to produce a desired output. In the case of classification, the system's goal is to determine which of a set of diagnoses is most likely given the input. The set of diagnoses will preferably include a null option that represents no disease or movement disorder. In certain embodiments, the output of a classification system is generally a probability associated with each possible diagnosis (where the probabilities across all output sum to 1). In a regression system, real valued outputs are predicted independently. For example, the system could be trained to predict scores that fall on an institutional scale for measuring the severity of a disorder (e.g., Unified Parkinson's Disease Rating Scale (UPDRS)). As will be apparent to those with skill in the art, machine learning classification/regression algorithms that might be used to produce the final output are artificial neural networks (relatively shallow or deep) (Goodfellow, I., Bengio, Y., and Courville, A. (2016). Deep Learning. The MIT Press.), recurrent neural networks, support vector machines (Hearst, M. (1998). Support Vector Machines. IEEE Intelligent Systems 13, 4 (July), 18-28.), and random forests. The system may also utilize an ensemble of machine learning methods to generate the output (Zhang, C. and Ma, Y. (2012). Ensemble Machine Learning: Methods and Applications. Springer Publishing Company.).

A range of sensors may be employed to collect data from the patient to be used as input to the machine learning system. By way of example and not limitation, sensors are discussed below along with examples of how the data from them may be processed. These examples are meant to illustrate the types of analyses that may be applied but does not cover the full range of analyses the system can include.

Image analysis (from video): Video analysis of the patient may include analysis of the patient's face and facial movements, mouth specific movements, arm movements, full body movement, gait analysis, finger tapping. The video camera will be positioned in a manner to completely capture the relevant content (e.g., if the focus is just the face, the camera will be close to the face but will not cut off any part of the face/head, or if the focus is the hand for finger tapping, just the patient's hand will be in frame). The system may aid the user in collecting the appropriate images by providing an on-screen prompt, such as a frame on the video display of the device. Given a video sequence of the specific body location being observed, initial processing may be done to accurately localize the body part and its sub components (e.g., the face and parts of the face such as eye and mouth locations). The localization may be used to constrain the region over which further processing and feature extraction is performed.

Audio analysis (from video or microphone): Throughout the course of video recording, the audio signal may also be recorded. Alternately, a microphone may be used to acquire audio data independently of a video. In some cases, when the focus is purely on movement, the audio data will not be used. However, in other aspects of the test, the audio signal may include speech from the patient or other sounds that are relevant to the task being performed and may provide diagnostic information (e.g., Zhang, Y. (2017). Can a Smartphone Diagnose Parkinson Disease? A Deep Neural Network Method and Telediagnosis System Implementation. Parkinson's Disease, vol. 2017.). Furthermore, the patient may be prompted to read a specific statement aloud to provide a standardized audio sample across all patients, or make repetitive plosive sounds (“PA,” “KA,” and “TA”) for a specific duration. In the case that the audio is being used, the processing may involve detection of speech and other sounds, statistical analysis of the audio data, filtering of the signal for feature extraction. The raw audio data and or any derived features could then be provided as input to a recurrent neural network to perform further feature extraction. Finally, the intermediate representation might be passed to another neural network to generate the desired output or could be combined with features from other modalities before passed to the final decision making component.

Range imaging system (e.g., Infrared Time-of-flight, LiDAR, etc.): Range imaging systems record information about the structure of objects in view. Typically they record a depth value for every pixel in the image (though in the case of LiDAR, they may produce a full 3D point cloud for the visible scene). 2D depth data or 3D point cloud data can be integrated into the machine learning system to assist in object localization, keypoint detection, motion feature extraction, and classification/regression decisions. In many instances, this data is processed in a similar manner to image and audio data in that it often requires preprocessing, normalization, and feature extraction.

Gyroscope and accelerometer: Most hand held devices (e.g., smartphones and tablets) include sensors that measure orientation and movement of the device. These sensors may be used by the machine learning system to provide supplemental diagnostic information. In particular, the sensors can be used to record movement information about the patient while he or she is performing a particular task. The movement data can be the primary source data for the task or can be combined with video data recorded at the same time. The temporal movement data can be processed in a similar way to the video data using preprocessing stages to prepare the data and feature extraction to obtain a discriminative representation that can be passed to the machine learning algorithm.

Touch screen/pressure sensors: Many devices have an onboard touch screen that captures physical interactions with the device. In some cases, the device also has more fine resolution pressure sensors that can differentiate between different types of tactile interactions. These sensors can be integrated into the machine learning system as an additional source of diagnostic information. For example, the patient may be directed to perform a sequence of tasks that involve interacting with the touch screen. The timing, location, and pressure of the patient's responses can be integrated as supplemental features in the machine learning system.

The machine learning system may be trained to produce the expected output for a given input set. In certain embodiments, expert neurologists who have viewed and annotated the raw input data will define the data outputs used in training the machine learning system. Alternately (or in addition), the outputs for some tests may be defined by information known about the patient. For example, if a patient is known to have a particular movement disorder, that information may be associated with the input of a particular test even if the expert neurologist cannot diagnose the movement disorder from that particular test alone. An annotated dataset covering a range of healthy and diseased patients will be assembled and used to train and validate the machine learning system. The artificial intelligence system may integrate additional expert knowledge that is not learned from the data but is deemed important for the diagnosis (for example, a supplemental decision tree (Quinlan, J. (1986). Induction of Decision Trees. Machine Learning 1 (1): 81-106.) defined by an expert neurologist).

The dataset will be generated in part from recordings performed on devices similar to those that will be used when the system is deployed. However, training may also rely on data generated from other sources (e.g., existing video recordings of patients with and without movement disorders).

Preferably, once the system is in operation additional data may be collected (with the patient's permission) and used to train and improve future versions of the machine learning system. This data may be recorded on the device and transferred to permanent computer storage at a later time or may be transmitted to off device storage system at real or near-real time. The means of transfer may include any commonly available wired or wireless technology.

In certain embodiments, a deep learning approach may be used to perform the desired classification/regression task. In this case, the deep learning system will internally generate an abstract feature representation relevant to the problem. In particular, the temporal data may be processed using a recurrent neural network such as a long short-term memory (LSTM), to obtain a deep, abstract feature representation. This feature representation may then be provided to a standard deep neural network architecture to obtain the final classification or regression outputs.

Turning now to the figures, a block diagram of one embodiment of the present invention is described. Figure one illustrates one example of how the Artificial Intelligence system of the present invention may be trained. First, the raw data (101) is acquired from a number of healthy individuals, as well as from individuals who have been diagnosed with the disease (or diseases) of interest. Such data may be collected from a number of different sensor types, including video, audio, or touch based sensors. Preferably, multiple different types of data will be collected from each sensor as described above. During the training process, the data will then be classified by experts trained in diagnosing the relevant disease (102). This classification may be specific to the test preformed (such as using the UPDRS scale for a specific task related to Parkinson's Disease), or it may be a simple binary designation relating to the patient's overall diagnosis, regardless of whether the specific test at issue is indicative of the disease.

This raw data will then undergo data processing (103). It will be apparent to those having skill in the art that the data processing may take place on the device used to collect the data, or the raw data may be transmitted to a remote server using any wired or wireless technology to be processed there. Also, it will be apparent that feature extraction may be performed as part of the data processing stage of the system, or may be performed by the machine learning system during the training and model generation stage, depending on the specific machine learning system used. Furthermore, it is possible that the classification step described in (102) above may be performed after the data is processed, rather than before.

Preferably, the system of the present invention will compare the subjects classified as having a particular neurological disorder to the subjects classified as “healthy” to facilitate training of the diagnostic models.

In certain embodiments, the sensor data may be processed using image processing, signal processing, or machine learning to extract measurements associated with some action (e.g., jaw displacement in tremor, finger tapping rate, repetitive speech rate, facial expression, etc.). These measurements can then be compared to normative values for healthy and diseased patients collected via the system or referenced in the literature for various disorders. As an example, a common speech test for Parkinson's Disease is to repeatedly say a syllable (e.g., “PA”) as many times as possible in 5 seconds. The system would record audio of a person completing this task and would use signal processing or machine learning methods to count the total number of utterances within the 5 second window. A diagnosis could be obtained by comparing the total utterance count to the distribution of counts observed across a population of healthy people. Additionally, the measurement could serve as a feature for a downstream machine learning system that learns to make a diagnosis from a collection of varying measurements perhaps combined with other features extracted from additional sensor data.

Once the data has been prepared, it is used to train a plurality of machine learning systems to generate a number of classification models (104) that, when combined, are used to produce a predictive diagnostic model. Preferably, each of the trained diagnostic models will focus on a single aspect (or subset of aspects) of the collected patient data. For example, diagnostic model 1 may focus exclusively on the blink rate of a video of the patient's face, while diagnostic model 2 may focus on the frequency of a repetitive finger tapping test. Preferably such diagnostic models will be trained by comparing the data from subjects which have been classified as possessing a certain neurological disorder to the data from subjects which have been classified as “healthy.” Preferably, a large number of such trained diagnostic models will be generated for each possible disease. Doing so will enable the overall system to accommodate instances where an individual test is inconclusive or missing. The classifications produced by these trained diagnostic models will then be aggregated (105) by an additional Artificial Intelligence (AI) system to produce a final predicative diagnostic model (106).

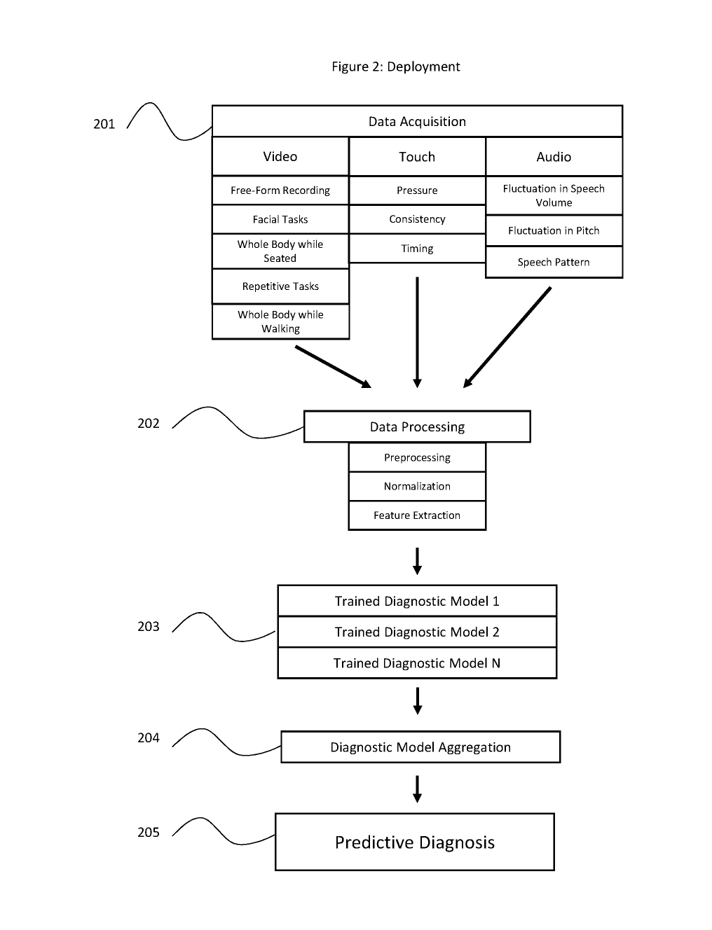

Upon deployment, the trained system may be used to produce a predictive diagnosis for a patient (FIG. 2). Preferably, the data acquisition (201) and processing (202) steps will be similar or identical to the methods used during the training of the diagnostic system. Once processed, the system will pass the data to the relevant trained diagnostic model, whereby each model will assign a classifier to the data based on the results of the training described above (203). The outputs of each diagnostic model will then be aggregated (204), and the system will thereby produce a predictive diagnostic output (205).

It will be apparent to those having skill in the art that, when deployed, the data acquisition, processing, training, and diagnosis steps can be performed on the device used to collect the data, or can be performed on different devices by transmitting the data from one device to another using any known wired or wireless technology.

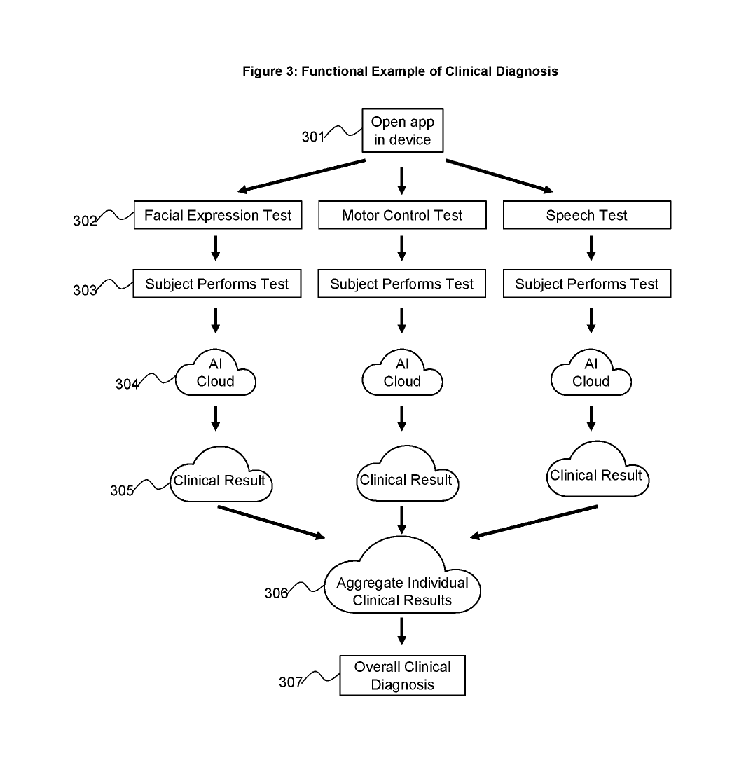

FIG. 3 illustrates one possible implementation the system of the present invention to diagnose a patient which may potentially have a neurological disorder. First, the user instructs a mobile device, such as a cell phone or tablet computer, to run an application that can execute the program of the present invention (301). The user is then prompted to perform a series of tests on the subject to be diagnosed (302). It will apparent that the user and the subject can be the same person, or different people. In this example, the application has prompted the user to perform three tests, one focusing on recording various facial expressions using the device's built-in camera, one focusing on fine motor control using an accelerometer equipped within the device, and focusing on speech patterns by having the user read a sentence displayed on the screen and recording the speech using the device's microphone. As the user performs the prompted tests, the relevant data is collected (303). In this example, the data is then transmitted to a remote cloud server, where a trained AI program of the present invention processes and analyzes the data (304) to produce a clinical result based on the particular test (305). The individual clinical results are then aggregated by a trained AI program (306) to produce a final clinical result (307) which is output to the user. It will be apparent to those having skill in the art that additional sensor inputs could also be used, and that any individual AI program could incorporate data from one or more sensors to produce an individual clinical result. It will further be apparent that the trained AI program could be housed on the device used to collect the data, provided the device has sufficient computing power an storage to run the full application.

### Working Example

The following Working Example provides one exemplary embodiment of the present invention, and is not intended to limit the scope of the invention in any way. This is one specific embodiment of a general system that diagnoses movement disorders. Such disorders include, but are not limited to, the following: Parkinson's Disease (PD), Vascular PD, drug induced PD, Multisystem atropy, Progressive Supranuclear Palsy, Corticobasal Syndrome, Front-temporal dementia, Psychogenic tremor, Psychogenic movement disorder, and Normal Pressure hydrocephalus; Ataxia, including Friedrichs Ataxia, spinocerebellar ataxias 1-14, X-linked congenital ataxia, Adult onset ataxia with tocopherol deficiency, Ataxia-telangiectasia, and Canavan Disease; Huntington's disease, Neuro-acanthocytosys, benign hereditary chorea, and Lesch-Nyan syndrome; Dystonia, including Oppenheim's torsion dystonia, X-linked dystonia-Parkinsonism, Dopa-responsive dystonia, Craio-cervical dystonia, Rapid onset dystonia parkinsonism, Niemann-Pick Type C, Neurodegeneration with iron deposition, spasmodic dysphonia, and spasmodic torticollis; Hereditary hyperplexia, Unverricht-Lundborg disease, Lafora body disease, myoclonic epilepsies, Creutzfeldt-Jakob Disease (familial and sporadic), and Dentatorubral-pallidoluysian atrophy (DRPLA); Episodic Ataxias 1 and 2, Paroxysmal dyskinesiase, including kinesigenic, non-kinesigenic, and exertional; Tourette's syndrome and Rett Syndrome; Essential tremor, primary head tremor, and primary voice tremor.

The training process involves six primary stages: 1) data acquisition, 2) data annotation, 3) data preparation, 4) training diagnostic models, 5) training model aggregation and 6) model deployment. Generally, multiple tests are used for diagnosing Parkinson's disease and as such, the details of these 5 stages may vary some from one test to another. The methods below utilize only data that can be collected via a standard video camera (e.g., on a smart phone or computer). However, data from other sensors could be added as extra input.

### 1. Data Acquisition

A range of tests may be recorded using a video camera with a functional microphone. The procedure for recording these data should be consistent from one patient to the next. These video recordings will be used for training models to diagnose PD and will serve as the input for the deployed system when making a diagnosis for a new patient. The preferred tests can be broken down into the following tests (some of which may require multiple recordings), although it will be apparent to those having skill in the art that fewer or alternate tests may also be performed while maintaining diagnostic accuracy:

Record close-up video of the patient's face while prompting a sequence of actions. The goal of this test is to collect video that contains the face at rest, the face performing simple expressions, blink rate information, and gaze variations (side-to-side, up-down, convergence).

Record video of the patient's whole body while the patient is seated. The goal of this test is to capture video that contains the patient's hands and feet in a rested position. The data will also contain video of the patient raising their arms and holding them straight in front of themselves.

Record close-up video (with audio) of the patient's face while they say a prompted sentence or perform an alternative method of speech analysis. The speech analysis may ask the patient to say repetitive plosive sounds (“PA”, “TA”, “KA”, and “PA-TA-KA” for a specified duration, or read aloud a paragraph.

Record multiple clips of the patient performing repetitive movements. These movements include finger tapping, opening and closing hand repetitively, hand rotations (pronate/supinate), heel tapping. In each case, the video will be zoomed in on the body part performing the action (i.e., for finger/hand movements, the hand should nearly fill the video frame and for foot movements, the foot should nearly fill the video frame).

Record the patient getting up from his or her chair, walking 10-15 steps, turning 180 degrees and walking back. This should be recorded in a way that captures a frontal view of the patient getting out of the chair. Additionally, the recording should include a frontal view of the patient at some point during the walking.

For the purpose of training diagnostic models, the above data will be recorded for a population of diseased and healthy individuals. Ultimately, recordings for a large population of individuals are desired. However, the dataset may grow iteratively with intermediate models being trained on available data. For example, the system could be deployed in a smart phone app that directs a patient to perform the above tests. The app could use existing trained models to offer a diagnosis for the patient and the data from that patient could then be added to the set of available training data for future models.

### 2. Data Annotation

Following data acquisition, a data annotation phase will be required for labeling properties of the video recordings. A trained expert will review each video recording and provide a collection of relevant assessments. When appropriate, the expert will assign a Unified Parkinson's Disease Rating Scale (UPDRS) rating for various observable properties of the patient. For example, for the face recording in Test 1, a UPDRS score will be assigned for facial expression and face/jaw tremor. For situations where the UPDRS is not applicable, the expert may assign an alternative label to the video recording. For example, for the face recording in Test 1, the expert may classify the patient's blink rate into 5 categories ranging from normal to severely reduced. For Test 2, the expert will assign a UPDRS score for the amount of tremor in each extremity. For Test 3, the expert will assign a UPDRS score for the patient's speech based on the number of plosive sounds a specific duration, or on the resonance, articulation, prosody, volume, voice quality, and articulatory precision of the prompted paragraph. For Test 4, the expert will assign a UPDRS score for each repetitive movement task performed. For Test 5, the expert will assign a UPDRS score for arising from the chair, posture, gait, and body bradykinesia/hypokinesia. The expert may identify and label any other discriminate properties of the video recordings that could assist in a diagnosis, such as muscle tone (rigidity, spasticity, hypotonia, hypertonia, dystonia and flaccidity) through video analysis of specific tasks, including alternating motion rate (AMRs) and gait analysis.

In addition to the expert annotations described above, the data may require other forms of non-expert annotation. Generally, these annotations are not concerned with diagnosing PD and are instead focused on labeling relevant properties of the video. Examples of this include: trimming the ends of a video recording to remove irrelevant data, marking the beginning and end of speech, identifying and labeling each blink in a video sequence, labeling the location of a hand or foot throughout a video sequence, marking the taps in a video of finger tapping, segmenting actions in the video from Test 5 (e.g., arising from chair, walking, turning), etc.

Consistent annotations should be provided for all of the data available for training models. For the diagnostic annotations (UPDRS or other classification), all training examples must be labeled. Non-diagnostic annotations may not be required for every training example as they will generally be used for training data preparation stages rather than for training the final diagnostic models.

### 3. Data Preparation

The raw video and audio data usually needs to go through several stages of preparation before it can be used to train models. These stages include data preprocessing (e.g., trimming video/audio, cropping video, adjusting audio gain, subsampling or supersampling time series, temporal smoothing, etc.), normalization (e.g., aligning audio clips to standard template, transforming face image to canonical view, detecting object of interest and cropping around it, etc.), and feature extraction (e.g., deriving Mel Frequency Cepstral Coefficients (MFCC) from acoustic data, computing optical flow features for video data, extracting and representing actions such as blinks or finger taps, etc.)

Given the data collected from the tests above, there are many different analyses that can be applied to obtain a final diagnosis. In what follows, examples of several such analyses are provided to illustrate the methods required to achieve a diagnosis in each case. In a final system, many diagnostic models (including those not described herein) would be trained and combined to achieve the overall diagnosis. The following examples were chosen to roughly cover methods appropriate for the first test described above. The various analyses within each of the 5 tests will generally exhibit more similarity. These same examples will be used in the subsequent section where the model training is described.

Face/Jaw Tremor Assessment (Data Preparation)

The data from Test 1 includes a close-up view of the patient's face at rest and performing some actions. This data could be used to identify and measure tremors in the jaw and other regions of the face. For simplicity here, we will assume that Test 1 was divided into sub collections and that the data available for this task contains a recording of only the face at rest.

In certain embodiments, the facial expression test asks the patient to observe a combination of video and audio that will likely illicit changes in facial expression. This may include (but are not limited to) humorous, disgusting or startling videos, or photographs with similar characteristics, or startling audio clips. While that patient is observing these stimuli. The camera (in “selfie mode,” or otherwise directed at the subject's face) is focused on the patient's face to analyze changes in facial expression and the presence or absence of jaw tremor.

The first stage in processing the raw video data is to find a continuous region(s) within the video where the face is present, unobstructed, and at rest. For this task, off-the-shelf face detection algorithms (e.g., Viola, Jones or more advanced convolutional neural networks) or those available via an online API such as Amazon Rekognition™ can be used to identify video frames where the face is present. Regions of the video where a face is not present will be discarded. If there are not enough continuous sections with the face present, the video will need to be re-recorded or the data will be discarded from the training set. The face detection algorithms run during this stage will also be used to crop the video to a region that only contains the face (with the face roughly centered). This process helps control for varying sizes of the face across different recordings.

The next step in face processing it to identify the locations of standard facial landmarks (e.g., eye corners, mouth, nose, jaw line, etc.). This can be done using freely licensed software or via online APIs. Alternatively, a custom solution for this problem can be trained using data from freely available facial landmark datasets.

Once the locations of key facial features are known, the algorithm extracts regions of interest from the video by cropping a rectangular region around a portion of the face. One such region includes the jaw area and extends roughly from slightly below the chin to the middle of the nose in the vertical direction and to the sides of the face in the horizontal direction. Other regions of the face where tremors occur may also be extracted at this point. Additionally, a crop of the whole face is may be retained.

During the extraction of the regions of interest, image stabilization techniques are used to assure a smooth view of the object of interest within the cropped video sequence. These techniques may rely on the change in the detected face box region from one frame to the next or similarly the change in the location of specific facial landmarks. The goal of this normalization is to obtain a clear, steady view of the regions of interest. For example, the view of the jaw region should be smooth and consistent such that a tremor in the jaw would be visible as up and down movement within the region of interest and would not result in jitter in the overall view of the jaw region.

At the end of this stage, the prepared data consists of a collection of videos that are zoomed in on specific views of the face. As a final processing step, the duration of these clips may be modified to achieve a standard duration across patient recordings.

### 4. Training Diagnostic Models

Once the raw video and audio data has been prepared using the techniques described above, models are trained to make accurate diagnostic decisions. Many different models would be trained to diagnose different aspects of the patient's movements. As in the previous section, several specific examples are described in detail here. However, those not described here would be similar in nature.

Furthermore, additional medical information not derived from the tests above could be used as a training input for the models. For example, relevant information such as the age, weight, medical history, or family history of the patient could be provided directly to the system of the present invention. Such information could be automatically extracted from the patient's Electronic Health Records, or entered manually by the patient or physician in response to a questionnaire presented by the system.

4.1. Face/Jaw Tremor Assessment (Model Training)

The dataset prepared according to the description above contains one or more video sequences of face regions of interest. These sequences have been standardized to include a fixed number of frames. Additionally, for each sequence, we have an expert annotation for the UPDRS score associated with the face/jaw tremor observed. For the sake of simplicity, we will describe a model for a single region of interest and then briefly discuss how this framework could be extended to multiple regions of interest.

Consider a video sequence of a jaw recorded at 30 frames per second for 10 seconds. Assumed that the cropped region around the jaw has a dimension of 128×256 pixels (rows×columns). The data would then be a sequence of 300 sample images each of size 128×256 (these numbers are merely for illustration purposes and do not reflect the exact dimensions used in the model). For each patient, we have such a sequence and an associated UPDRS score for that patient. The goal of training a model is to learn to predict the UPDRS score from the input sequence derived from the data.

To learn this mapping, we use a combination of convolutional neural networks and recurrent neural networks (in particular Long short-term memory (LSTM) networks). We define a standard collection of convolutional blocks that operate on the independent image frames. Each block includes a combination of convolutional operators and optional pooling and normalization layers. The blocks may also include skip connections that feed the input data or a modified version of it forward in the network. At the end of the convolutional blocks, the features are flattened into a single feature vector. The model learns the weights of the convolutional blocks so as to generate a single feature vector for each image that is useful for the discriminative task at hand. At this point in the network processing pipeline, there is a feature vector for each image frame in the video sequence. This sequence of features is passed to an LSTM network that learns to integrate across the temporal dimension in the data. The LSTM network in turn generates a feature vector for the whole sequence that can be used for generating a final real-valued prediction for the UPDRS score. Learning in the network is performed by back propagating the loss associated with the predicted UPDRS score up through the LSTM layer and then through the convolutional blocks using standard optimization methods such as stochastic gradient descent. It should be noted that the above description is just a sketch of one such model that could be applied to this problem and there are many reasonable variants to it that could be equally effective. Implementation, training and deployment of such a network can be achieved using standard neural network libraries such as TensorFlow, Caffe, etc.

The description above is of a model that operates over a single region of interest. However, the technique generalizes to multiple regions of interest and a whole model operating on all regions can be trained in one pass. The general approach is to run several of these models concurrently to generate a prediction or feature representation for each of the regions of interest. These predictions or features can then be combined in the network architecture and used via a final fully connected network to make an overall UPDRS score prediction. The learning error can propagate from this final end prediction up through all of the branches of the model associated with specific regions of interest.

### 5. Training Model Aggregation

The goal of a general system for diagnosing PD is to produce a final diagnosis for a patient or to provide an overall UPDRS score for the patient. In order to do this, a final model must be trained to learn how to aggregate the predictions from the set of models that are trained to identify particular movement abnormalities.

As input for the final model, we have the predictions from each intermediate model that may be real-values scores, ordinal classifications or general classifications. In addition to these predictions, we may have confidence values for the predictions and other relevant outputs from the intermediate models. For each patient, we assume that we have an expert annotation for the overall UPDRS score for that patient.

A standard random forest regression model is trained to predict the overall UPDRS score from the input data. Such a model can be trained and deployed using standard machine learning libraries such as scikit-learn. Many different models could be used to learn to make the overall diagnosis and random forest regression is suggested as just one example.

### 6. Model Deployment

When deploying this system for diagnosing PD, the same data acquisition process would be applied for a given patient. There would be no annotation of the data as the goal is for the system to perform this. The raw data would be prepared according to the methods in Section 3 above, and would be passed on to the trained models described in Section 4 (though no actual training would be done at this stage). The output of each of the trained diagnostic models would then be passed to the final model to make the overall diagnostic prediction. The predictions from the intermediate models may also be made available in the final diagnosis.

As an example, such a system could be implemented in a smart phone app. Data for the patient would be collected by following a process within the app that records video and prompts for the appropriate patient actions. The app would cycle through a series of discrete tests that correspond roughly to the tests above (though some of the above tests would be divided into multiple subtests). Data from each test would be saved on the device or uploaded to the cloud. Additionally, the data would be passed to the appropriate data preparation methods that in turn would pass the prepared data to the appropriate diagnostic model. The data from a single test might be passed to multiple different diagnostic pipelines (consisting of data preparation and model evaluation). The diagnostic pipelines may be implemented on device, on a remote computer, or some combination of both. Once all of the diagnostic models have been run, their output would be passed to the final model to obtain the overall diagnostic prediction. Again, this processing could be done on device, in the cloud, or some combination of both. The system would output the final diagnostic prediction to the patient along with intermediate model predictions. The system may display such an output on the screen of the device used to collect the initial senor data, or may transmit it to the relevant parties via other means, such as SMS messaging to a mobile device or sending an email to a designated party. The system might present additional information relevant to the diagnostic prediction (e.g., confidence scores, assessment of recording quality, recommendations for follow up tests, etc.). The app may also log relevant information and data from the tests and could pass along information regarding the diagnosis to a selected medical professional.

In addition to the working example relating to movement disorders presented above, the system of the present invention would also be applicable to diagnosing the following diseases, as well as many others.

Stroke

In one embodiment, the artificial intelligence system will autonomously decide on whether tissue plasminogen activator (tPA) or (“clot buster”), or other treatment such as endovascular treatment or use of an antithombotic treatment, is appropriate to deliver to patients presenting with a stroke emergency. The patient presenting with acute stroke symptoms will be evaluated simultaneously by the emergency physician and the Acute Stroke Artificial Intelligence System (ASAIS). The ASAIS will have at least one of three general types of sensors to assess the patient, including video, audio, and infrared generator/sensor. In addition, there will be ‘clinical data’ input. The clinical data input can be manually entered by a nurse or medical assistant OR be linked with the facilities electronic health record (EHR) for direct transfer of some of the data. The clinical data includes: biographic data, time of onset of symptoms or last time the patient was seen as ‘normal’, laboratory data (platelet count, international normalized ratio and prothrombin time), brain imaging data (typically head computed tomogram without contrast) and blood pressure. Lastly, there will be a brief set of ‘yes/no’ questions that are required and will need to be manually entered. These will include:
1. Any KNOWN internal bleeding—yes or no
2. Any KNOWN history of recent (within 3 months) of intracranial or intraspinal surgery? Or serious head trauma?—yes or no
3. Any KNOWN intracranial conditions that may increase the risk of bleeding?—yes or no
4. Any KNOWN bleeding diathesis?—yes or no
5. Any KNOWN arterial puncture at a non-compressible site within the last 7 days? yes or no

In certain embodiments, the sensors will determine factors including, but not be limited to, detection of patient signs relevant to the assessment of each aspect of the modified National Institutes of Health Stroke Scale (mNIHSS). Such tests include the following:

Horizontal eye movement, distinguishing between normal movement, partial gaze palsy and total gaze paresis.

Visual field assessment, distinguishing among normal visual field, partial hemianopia or complete quadrantanopia; patient recognizes no visual stimulus in one specific quadrant versus complete hemianopia; patient recognizes no visual stimulus in one half of the visual field; and total blindness.

Motor arm assessment for both left and right arms independently, distinguishing among no arm drift; arm remains in the initial position for 10 seconds, drift; the arm drifts to an intermediate position prior to the end of the full 10 seconds, but not at any point relies on a support, limited effort against gravity; the arm is able to obtain the starting position, but drifts down from the initial position to a physical support prior to the end of the 10 seconds, no effort against gravity; the arm falls immediately after being helped to the initial position, however the patient is able to move the arm in some form (e.g. shoulder shrug), and no movement; patient has no ability to enact voluntary movement in this arm.

Motor leg assessment for both left and right legs independently, distinguishing among no leg drift; if remains in the initial position for 5 seconds, drift; the leg drifts to an intermediate position prior to the end of the full 5 seconds, but at no point touches the bed for support, limited effort against gravity; the leg is able to obtain the starting position, but drifts down from the initial position to a physical support prior to the end of the 5 seconds, no effort against gravity; the leg falls immediately after being helped to the initial position, however the patient is able to move the leg in some form (e.g. hip flex), and no movement; patient has no ability to enact voluntary movement in this leg.

Language assessment, distinguishing among normal speech, mild-to-moderate aphasia; detectable loss in fluency, but some information content severe aphasia; all speech is fragmented, and the patient's speech has no discernable information content, and patient is unable to speak.

Dysarthria assessment, having the patient read from the list of words provided with the stroke scale and distinguishing between normal; clear and smooth speech, mild-to-moderate dysarthria; some slurring of speech, however the patient can be understood, and severe dysarthria; speech is so slurred that he or she cannot be understood, or patients that cannot produce any speech

Assessment of extinction and inattention, distinguishing among normal, inattention on one side in one modality; visual, tactile, auditory, or spatial and hemi-inattention; does not recognize stimuli in more than one modality on the same side.

This aggregate data will then be analyzed by the ASAIS. The collection component of ASAIS may be locally housed in a laptop with software being stored/operated via cloud technology. In one embodiment, the ASAIS decision making algorithms will generate one of three ultimate outputs: YES, NO or MAYBE to administering tPA to the patient. The emergency physician can use his own judgement along with the output with the ASAIS to make a final decision to whether to give tPA or not. Flow chart 1 shows this basic process.

It is very important to note, that currently due to significant shortages in neurologists, there is pervasive use of telemedicine in many emergency departments across the US. Therefore, the ASAIS could be embedded within an existing teleneurology service to further scale up the neurologists volume of hospitals covered (within limits) and provide a human neurologist ‘back-up’ for any cases that are deemed uncertain by the emergency physician.

In the preferred embodiment, there are three possible outputs from the ASAIS: YES, NO and MAYBE. One output is YES to administering tPA to the patient. If the emergency physician agrees with the output, tPA will be administered. If the emergency physician questions or is uncertain of the output, a remote neurologist may use telemedicine technology to be directly involved in the case and give the final recommendation. The second output is NO to administering tPA. In this case, the neurologist will be directly involved in only those cases in which the emergency physician questions or is uncertain of the output, as outlined above. The third output option is MAYBE to administering tPA. The neurologist will be involved in all of these cases via telemedicine.

In addition to the primary ultimate outputs (YES, NO and MAYBE to tPA administration) there may also be a simultaneous modified National Institutes of Health Stroke Scale (mNIHSS) output for physician utilization. The National Institutes of Health Stroke Scale (NUBS) is a standardized neurologic exam scale used widely to rate severity of stroke deficits. The range is from 0 (normal) to 42 (most severe stroke). In broad terms, 0-5 scores of the NIHSS correlate to small strokes and scores above 20 and above correlate to large strokes. Due to anticipated technical limitations, the NIHSS may be modified.

In an alternate embodiment, the invention will have a mobile application version for home self-testing use. This application will utilize the video, audio and, if available on the device, infrared time-of-flight.

Neurostimulation Device Calibration

Neurostimulation devices are medical devices that provide electrical current to specific regions of the brain or other parts of the nervous system for a therapeutic effect. In movement disorders, one variant of such neurostimulation devices are termed deep brain stimulation (DBS) devices, such as those described in U.S. Pat. No. 8,024,049. DBS is a FDA approved therapy for Parkinson's Disease, tremor and dystonia. In the future, DBS will likely gain FDA approval for stroke recovery. The first DBS implant for stroke recovery occurred on Dec. 19, 2016 at the Cleveland Clinic (Ohio) using a device produced by Boston Scientific.

It will be apparent to those having skill in the art that such implanted medical devices require special programing to ensure that the device behaves appropriately and provides the optimal outcome for the patient. As such, each implanted device must be specifically calibrated to the patient to maximize its therapeutic effect. Currently, the best practices for programming a DBS (both initially and during follow-up visits) involve a significant amount of trial and error, which results in significant uncertainty for the patient, and has the potential to result in sub-optimal outcomes. See Picillo et. al. (2016), Programming Deep Brain Stimulation for Parkinson's Disease: The Toronto Western Hospital Algorithms, Brain Stimulation 9(3), 425-437. As such, there is a need for a system that can make accurate programming recommendations for a patient.

As such, in certain embodiments of the present invention, the system of the present invention may be used to produce specific programing suggestions to optimize the performance of the implanted device in the patient to both improve therapeutic efficacy, such as, but not limited to, improving rigidity, tremor, akinesia/bradykinesia or induction of dyskinesia, and reduce unintended side effects such as, but not limited to, dysarthria, tonic contraction, diplopia, mood changes, paresthesia, or visual phenomenon of the device.

Utilizing the sensor and diagnostic system of the present invention, the sensor inputs described in the working example above, preferably including facial expression, motor control, and speech pattern diagnostics, may be used to train a machine learning algorithm to make specific suggestions regarding the various programing variables available on DBS devices. Such suggestions include changes in AMPLITUDE (in volts or mA), PULSE WIDTH (in microseconds {usec}), RATE (in Hertz), POLARITY (of electrodes), ELECTRODE SELECTION, STIMULATION MODE (unipolar or bipolar), CYCLE (on/off times in seconds or minutes), POWER SOURCE (in amplitude) and calculated CHARGE DENSITY (in uC/cm2 per stimulation phase).

Once trained, the system of present invention may use similar data collected from individual patients to make specific recommendations for altering the programing variables for each patient's implanted device.

One key benefit of the system of the present invention is that such programming changes may be made in real time, with the system monitoring the patent to both validate any suggested programming changes or potentially suggest additional changes that may further improve the function of the medical device for the patient.

Thus, in certain embodiments the sensor data may be analyzed in real time by machine learning and optimization systems through an iterative process testing a large number (thousands to millions) of possible DBS stimulation patterns via direct communication with the implanted pulse generator (IPG) through standard telemetry, radiofrequency signals, Bluetooth™ or other means of wireless communication between the application and the IPG. The system finds the optimized DBS stimulation pattern and is able to set this stimulation pattern as a baseline. This baseline DBS stimulation pattern can be modified anytime manually by the healthcare provider-programmer or using this application for optimization at a later time. In further embodiments, the system of the present invention may use the same iterative process, described above to optimize stimulation patterns for other neuropsychiatric disorders, including obsessive-compulsive disorder, major depressive disorder, drug-resistant epilepsy, central pain and cognitive/memory disorders.

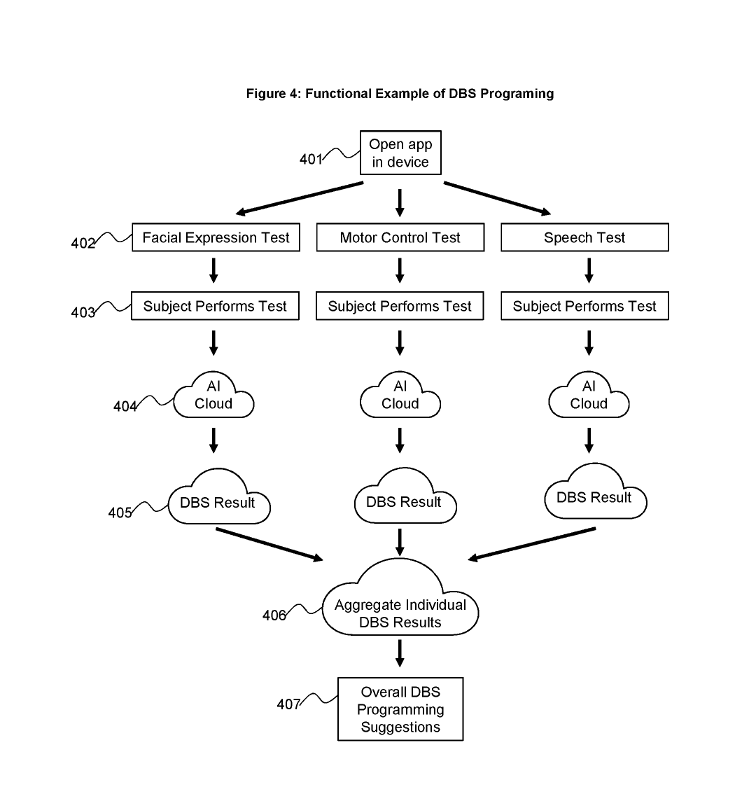

FIG. 4 illustrates one possible implementation the system of the present invention to produce recommendation for programing a DBS in a patient. First, the user instructs a mobile device, such as a cell phone or tablet computer, to run an application that can execute the program of the present invention (401). The user is then prompted to perform a series of tests on the subject to be diagnosed (402). It will apparent that the user and the subject can be the same person, or different people. In this example, the application has prompted the user to preform three tests, one focusing on recording various facial expressions using the device's built-in camera, one focusing on fine motor control using an accelerometer equipped within the device, and focusing on speech patterns by having the user read a sentence displayed on the screen and recording the speech using the device's microphone. As the user performs the prompted tests, the relevant data is collected (403). In this example, the data is then transmitted to a remote cloud server, where a trained AI program of the present invention processes and analyzes the data (404) to produce a DBS result based on the particular test (405). The individual DBS results are then aggregated by a trained AI program (406) to produce a final DBS result (407) which is output to the user, such as suggested programing settings for the variables described above. It will be apparent to those having skill in the art that additional sensor inputs could also be used, and that any individual AI program could incorporate data from one or more sensors to produce an individual clinical result. It will further be apparent that the trained AI program could be housed on the device used to collect the data, provided the device has sufficient computing power an storage to run the full application. Dizziness:

The role of this invention is to aid the physician, in any clinical setting, to help diagnose the cause of dizziness. The invention includes an Artificial Intelligence based system that uses video, audio and (if available) infrared time-of-flight INPUTS to analyze the patients motor activity, movements, gait, eye movements, facial expression and speech. It will also have inputs regarding the temporal profile of the dizziness (acute severe dizziness, recurrent positional dizziness or recurrent attacks of nonpositional dizziness). This data can be entered manually by a medical assistant or via natural language processing by the patient via prompts.

Seizures

The purpose of the invention is to aid in the differentiation of ES and NBS using machine learning algorithms primarily analyzing digital video. In other embodiments, additional inputs may also be utilized.

Preferably, the software can be embedded within existing infrastructure of EMUs and will have mobile/tablet version for patient home use. This will help motivate patients to record the events. In addition to having the analysis from the invention, they will able to share the video with their neurologist for confirmation.

Methods and components are described herein. However, methods and components similar or equivalent to those described herein can be also used to obtain variations of the present invention. The materials, articles, components, methods, and examples are illustrative only and not intended to be limiting.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art.

Having illustrated and described the principles of the invention in exemplary embodiments, it should be apparent to those skilled in the art that the described examples are illustrative embodiments and can be modified in arrangement and detail without departing from such principles. Techniques from any of the examples can be incorporated into one or more of any of the other examples. It is intended that the specification and examples be considered as exemplary only, with a true scope and spirit of the invention being indicated by the following claims.

We claim:

1. A system for diagnosing a neurological disorder in a patient, the system comprising:
i. at least one sensor in communication with a processor and a memory;
a. wherein said at least one sensor in communication with a processor and a memory acquires raw patient data from said patient;
i. wherein said raw patient data comprises at least one of a video recording and an audio recording;
ii. a data processing module in communication with the processor and the memory;
a. wherein said data processing module converts said raw patient data into processed diagnostic data;
iii. a diagnosis module in communication with the data processing module;
a. wherein said diagnosis module comprises a trained diagnostic system;
i. wherein said trained diagnostic system comprises a plurality of diagnostic models;
1. wherein each of said plurality of diagnostic models comprise a plurality of algorithms trained to assign a classification to at least one aspect of said processed diagnostic data; and
ii. wherein said trained diagnostic system integrates said classifications of said plurality of diagnostic models to output a diagnostic prediction for said patient.

2. The system of claim 1, wherein the program executing said diagnosis module is executed on a device that is remote from the at least one sensor.

3. The system of claim 1, wherein said trained diagnostic system is trained to diagnose a movement disorder.

4. The system of claim 3, wherein said movement disorder is Parkinson's Disease.

5. The system of claim 3, wherein said raw patient data comprises a video recording, wherein said video recording comprises at least one of: a recording of the patient's face while preforming simple expressions; a recording of the patient's blink rate; a recording of the patient's gaze variations; a recording of the patient while seated; a recording of the patient's face while reading a prepared statement; a recording of the patient preforming repetitive tasks; and a recording of the patient while walking.

6. The system of claim 3, wherein said raw patient data comprises an audio recording, wherein said audio recording comprises at least one of: a recording of the patient repeating a prepared statement; a recording of the patient reading a sentence; and a recording of the patient making plosive sounds.

7. The system of claim 1, wherein said plurality of algorithms are trained using a machine learning system.

8. The system of claim 7, wherein said machine learning system comprises at least one of: a convolutional neural network; a recurrent neural network; a long-term short-term memory network; support vector machines; and a random forest regression model.

9. A system for calibrating an implanted medical device in a patient, the system comprising:
i. at least one sensor in communication with a processor and a memory;
a. wherein said at least one sensor in communication with a processor and a memory acquires raw patient data from said patient;
i. wherein said raw patient data comprises at least one of a video recording and an audio recording;
ii. a data processing module in communication with the processor and the memory;
a. wherein said data processing module converts said raw patient data into processed calibration data.
iii. a calibration module in communication with the data processing module;
a. wherein said calibration module comprises a trained calibration system;
i. wherein said trained calibration system comprises a plurality of calibration models;
1. wherein each of said plurality of calibration models comprise a plurality of algorithms trained to assign a classification to at least one aspect of said processed calibration data; and
ii. wherein said trained calibration system integrates said classifications of said plurality of calibration models to output a calibration recommendation for said implanted medical device of said patient.

10. The system of claim 8, wherein the program executing said calibration module is executed on a device that is remote from the at least one sensor.

11. The system of claim 8, wherein said implanted medical device comprises a deep brain stimulation device (DBS).

12. The system of claim 10, wherein said calibration recommendation comprises a change to the programming settings of said DBS comprising at least one of: amplitude, pulse width, rate, polarity, electrode selection, stimulation mode, cycle, power source, and calculated charge density.

13. The system of claim 8, wherein said raw patient data comprises a video recording, wherein said video recording comprises at least one of: a recording of the patient's face while preforming simple expressions; a recording of the patient's blink rate; a recording of the patient's gaze variations; a recording of the patient while seated; a recording of the patient's face while reading a prepared statement; a recording of the patient preforming repetitive tasks; and a recording of the patient while walking.

14. The system of claim 8, wherein said raw patient data comprises an audio recording, wherein said audio recording comprises at least one of: a recording of the patient repeating a prepared statement; a recording of the patient reading a sentence; and a recording of the patient making plosive sounds.

15. The system of claim 8, wherein said plurality of algorithms are trained using a machine learning system.

16. The system of claim 15, wherein said machine learning system comprises at least one of:
a convolutional neural network; a recurrent neural network; a long-term short-term memory network; support vector machines; and a random forest regression model.

17. A system for monitoring the progression of a neurological disorder in a patient diagnosed with such a disorder, the system comprising:
i. at least one sensor in communication with a processor and a memory;
a. wherein said at least one sensor in communication with a processor and a memory acquires raw patient data from said patient;
i. wherein said raw patient data comprises at least one of a video recording and an audio recording;
ii. a data processing module in communication with the processor and the memory;
a. wherein said data processing module converts said raw patient data into processed diagnostic data;
iii. a progression module in communication with the data processing module;
a. wherein said progression module comprises a trained diagnostic system;
i. wherein said trained diagnostic system comprises a plurality of diagnostic models;
1. wherein each of said plurality of diagnostic models comprise a plurality of algorithms trained to assign a classification to at least one aspect of said processed diagnostic data;
ii. wherein said trained diagnostic system integrates said classifications of said plurality of diagnostic models to generate a current progression score for said patient; and
iii. wherein said progression module compares said current progression score for said patient to a progression score from said patient generated at an earlier timepoint to create a current disease progression state, and output said disease progression state.
